(19) United States
(12) Patent Application Publication
(10) Pub. No.: US 2023/0110021 A1
(43) Pub. Date: Apr. 13, 2023

(54) PREPARATION METHOD AND APPLICATION OF ION-ACTIVATED BIOADHESIVE HYDROGEL

(71) Applicant: Eye Institute of Shandong First Medical University, Qingdao (CN)

(72) Inventors: Weiyun SHI, Qingdao (CN); Qingjun ZHOU, Qingdao (CN); Long ZHAO, Qingdao (CN); Zhen SHI, Qingdao (CN)

(21) Appl. No.: 17/938,238

(22) Filed: Oct. 5, 2022

Related U.S. Application Data
(63) Continuation of application No. PCT/CN2022/073918, filed on Jan. 26, 2022.

(30) Foreign Application Priority Data
Oct. 13, 2021 (CN) 202111191132.3

(51) Int. Cl. A61L 24/00; A61L 24/02; A61L 24/04; A61L 24/10

(52) U.S. Cl. CPC A61L 24/0031; A61L 24/02; A61L 24/043; A61L 24/108

(57) ABSTRACT

A composite hydrogel contains alginate hydrogel and acellular matrix hydrogel. The ingredients used to produce the acellular matrix hydrogel contains acellular matrix and transglutaminases (TGases). The alginate hydrogel contains salginate complex. The alginate hydrogel and acellular matrix hydrogel constitute a three-dimensional crosslinking structure. The TGases can catalyze isopeptide bonding between acellular matrixes, and thus forms acellular matrix hydrogel through crosslinking.

### CROSS-REFERENCE TO RELATED APPLICATIONS

This application is a continuation of PCT International Application No. PCT/CN2022/073918, filed on Jan. 26, 2022, which claims the benefit of priority to Chinese Application No. 202111191132.3, filed on Oct. 13, 2021, and entitled “a composite hydrogel and preparation method and application thereof”, the content of each is incorporated herein by reference in its entirety.

### TECHNICAL FIELD

The present invention falls in the field of hydrogel preparation, specifically relates a composite hydrogel, preparation method and application thereof.

### BACKGROUND

The cornea is a transparent tissue on the ocular surface. It is vulnerable to trauma and infection, which may cause irreversible loss of corneal transparency and ultimately blindness. Traditional cornea repair is mainly treated with donor cornea transplantation, the operation of which is highly expensive. Hydrogel is a three-dimensional network polymer with high water content and good biocompatibility. The structure of hydrogel is similar to that of body tissue. It does not affect the metabolic process of living organisms, and metabolites can also be eliminated through hydrogel. Therefore, hydrogel is often used as a substitute for human organs and tissues.

At present, hydrogels which are applied to corneal repair include acrylate hydrogel, fibrin glue and polyethylene glycol gel. Although the mentioned materials can effectively seal corneal perforation as a substitute for corneal transplantation, they have poor adhesion and fall off easily. For example, the Chinese patent Publication No. cn109575316a discloses a hydrogel obtained by radical polymerization using N-acryloyl dry ammonia amide and hydroxyethyl acrylamide, which can be used as a substitute for natural vitreum. However, it lacks adhesion and has certain limitations when used in corneal repair.

### SUMMARY OF THE INVENTION

The purpose of the invention is to provide a composite hydrogel and its preparation and application. The composite hydrogel provided by the invention is characterized in excellent adhesion.

As to achieve the above purposes, the technical solutions are provided as follows: The present invention provides a composite hydrogel which comprises alginate hydrogel and acellular matrix hydrogel;

The ingredients used in preparing said acellular matrix hydrogel comprises acellular matrix and transglutaminases;

Said alginate hydrogel comprises alginate complex;

Said alginate hydrogel and acellular matrix hydrogel constitute a three-dimensional crosslinking structure.

Optionally, the mass ratio of alginate hydrogel and acellular matrix hydrogel is 1:1-4;

The mass of said alginate hydrogel is calculated with the mass of alginates; the mass of said acellular matrix hydrogel is calculated with the mass of acellular matrix.

Optionally, said alginate complex comprises alginate and metal cations, said alginates comprises one or more of unmodified alginates, peptide modified alginates and dopamine modified alginates.

Optionally, said metal cations comprises one or more of calcium ions, magnesium ions and zinc ions.

The invention also provides a preparation method of the composite hydrogel disclosed in the above technical solutions, which comprises:

After the mixing of acellular matrix solution, alginate solution, TGase, and buffer solution, using metal cation solution to promote three-dimensionally crosslink and then said composite hydrogel is obtained.

Optionally, mass concentration of said acellular matrix solution is 1-3%;

Mass concentration of said alginate solution is 2-10%.

Volume ratio of said acellular matrix solution and alginate solution is 1-5:1.

Optionally, the amount ratio of said TGase and acellular matrix in the acellular matrix solution is 20-100 U: 1 g;

Optionally, said buffer solution comprises phosphate buffer solution or trimethylaminomethane buffer solution;

The concentration of said buffer solution is 10-100 mmol/L;

The pH of said buffer solution is 6-8;

The volume of buffer solution accounts for 1-5% of the volume of the acellular matrix solution.

Optionally, the molar concentration of said metal cations is 0.2-5 mol/L.

The volume of said metal ion solution accounts for 0.5-1% of the volume of alginate solution.

The present invention also provides applications in donor cornea preparation, corneal adhesive, hydrogel dressing or drug carrier using said composite hydrogel or composite hydrogel prepared by said technical solutions.

The invention provides a composite hydrogel, which comprises alginate hydrogel and acellular matrix hydrogel; the ingredients used to produce said acellular matrix hydrogel comprises acellular matrix and transglutaminases (TGases); said alginate hydrogel comprises alginate complex; said alginate hydrogel and acellular matrix hydrogel constitute a three-dimensional crosslinking structure.

Said TGases in the invention can catalyze isopeptide bonding between acellular matrixes, and thus forms acellular matrix hydrogel through crosslinking; said TGases meanwhile catalyze isopeptide bonding between acellular matrix and cornea stroma, which increases the adhesiveness between acellular matrix and cornea stroma; in addition, alginate hydrogel and acellular matrix hydrogel intertwines with each other to form a three-dimensional crosslinking structure with high density and stability, which further enhanced the adhesiveness of the composite hydrogel synergistically.

### BRIEF DESCRIPTION OF THE DRAWINGS

FIG. 1 shows the SEM (scanning electron microscope) observation of the composite hydrogel in Example 1;

FIG. 2 shows fluorescent staining of living and dead cells in the composite hydrogel in Example 1;

FIG. 3 shows the optical coherence tomography of corneal repair area in Application Example 1;

FIGS. 4a and 4b show the maximum intraocular pressure that the corneal repair area can bear in Application Example 1 and Contrast Example 1;

FIG. 5 shows the gross observation of cornea repair area in Application Example 1;

FIG. 6 shows the fluorescein sodium staining of the cornea repair area in Application Example 1;

FIG. 7 shows the corneal nerve staining of the cornea repair area in Application Example 1.

### DETAILED DESCRIPTION OF THE EMBODIMENTS

The present invention provides a composite hydrogel which comprises alginate hydrogel and acellular matrix hydrogel;

The ingredients used in preparing said acellular matrix hydrogel comprises acellular matrix and transglutaminases;

Said alginate hydrogel comprises alginate complex;

Said alginate hydrogel and acellular matrix hydrogel constitute a three-dimensional crosslinking structure.

In the present invention, the mass ratio of alginate hydrogel and acellular matrix hydrogel is 1:1-4, further preferably 1: 2-3; the mass of said alginate hydrogel is calculated with the mass of alginates; the mass of said acellular matrix hydrogel is calculated with the mass of acellular matrix.

In the present invention, said alginate hydrogel comprises alginate complex. In the present invention, said alginate complex comprises alginate and metal cations. In the present invention, said alginates comprises one or more of unmodified alginates, peptide modified alginates and dopamine modified alginates; said unmodified alginates comprises is further preferably unmodified sodium alginate; said peptide modified alginates is further preferably peptide-modified sodium alginate; said dopamine modified alginates is further preferably dopamine-modified sodium alginate; when the alginates are more than two of the above-mentioned specific selections, the present invention does not specifically limit the ratio of the specific substances, and any ratio can be used for mixing.

In the present invention, said peptide modified alginates are preferably the peptide-modified alginates obtained by amidation reaction of polypeptides and alginates. The present invention does have no special requirements on the type of the polypeptides, and those known to the skilled in the art may be used.

In the present invention, the dopamine modified alginates are preferably the dopamine-modified alginates obtained by amidation reaction of dopamine and alginates. In the present invention, the catechol group on the dopamine-modified alginates can be chemically cross-linked with the corneal stroma, which further improves the adhesion performance of the composite hydrogel.

In the present invention, said metal cations comprise one or more of calcium ions, magnesium ions and zinc ions. When the metal cations are two or more of the above specific selections, there is no particular limitation on the ratio of the specific substances, and any ratio can be used for mixing.

In the present invention, the ingredients used in preparing said acellular matrix hydrogel comprises acellular matrix and transglutaminases; said acellular matrix preferably comprises human-derived or animal-derived acellular matrix; said human-derived acellular matrix is preferably human corneal acellular matrix or human amniotic membrane acellular matrix; said animal-derived acellular matrix is preferably porcine corneal acellular matrix. In the present invention, the dosage ratio of the transglutaminase and the acellular matrix is preferably 20-100 U:1 g, more preferably 30-90 U:1 g, and further more preferably 40-80 U:1 g.

The invention also provides a preparation method of the composite hydrogel disclosed in the above technical solutions, which comprises:

After the mixing of acellular matrix solution, alginate solution, TGase, and buffer solution, using metal cation solution to preform three-dimensionally crosslink and then said composite hydrogel is obtained.

In the present invention, without specific description, said ingredients are all commercially available products and well known to those skilled in the art.

In the present invention, the acellular matrix in the acellular matrix solution is the same as the acellular matrix described in the above technical solutions, and details are not repeated here. In the present invention, the mass concentration of said acellular matrix solution is preferably 1-3%, more preferably 1.5-2.5%, and further more preferably 1.8-2.3%.

In the present invention, the alginate in the alginate solution is the same as the alginate described in the above-mentioned technical solution, and details are not repeated here. In the present invention, the mass concentration of the alginate solution is preferably 2-10%, more preferably 3-9%, and further more preferably 4-8%.

In the present invention, the volume ratio of the acellular matrix solution and the alginate solution is preferably 1-5:1, more preferably 1.5-4.5:1, and further more preferably 2-4:1.

In the present invention, said buffer solution preferably comprises phosphate buffer solution or TRIS buffer solution. In the present invention, said phosphate buffer solution preferably comprises PBS buffer. In the present invention, the concentration of said buffer solution is preferably 10-100 mmol/L, more preferably 20-80 mmol/L, and further more preferably 30-70 mmol/L. In the present invention, the pH value of said buffer solution is preferably 6-8, more preferably 6.2-7.8, and further more preferably 6.4-7.6. In the present invention, the volume of said buffer solution preferably accounts for 1-5% of the volume of the acellular matrix solution, more preferably 2-4%, and further more preferably 3%. In the present invention, the buffer solution can improve the solubility of transglutaminase.

In the invention, the amount ratio of said TGase and acellular matrix in the acellular matrix solution is preferably 20-100 U:1 g, more preferably 30-90 U:1 g, and further more preferably 40-80 U: 1 g. In the invention, said TGase is characterized in excellent adhesion, it can also form peptide bonds between acellular matrixes in said acellular matrix solution and then crosslinks to form acellular matrix hydrogel, so that the acellular matrix hydrogel has excellent adhesion.

In the invention, said mixing process is preferred as: dissolving TGase in the buffer solution, then mixing with acellular matrix solution and alginate solution. There are no special limitations on said dissolving and mixing, the process can be carried out based on routines that well known by the skilled in the art.

In the invention, the molar concentration of metal cations in said metal cation solution is preferably 0.2-1 mol/L, more preferably 0.3-0.9 mol/L, and further more preferably 0.4-0.8 mol/L. In the invention, said metal cations are the same with those in the above technical solutions, and details are not repeated here. In the invention, said metal cation solutions optionally comprise calcium ion solution, magnesium ion solution or zinc ion solution. In the invention, the volume ratio of said metal cation solution accounts for in the alginate solution is 0.5-1%, more preferably 0.6-0.9%, and further more preferably 0.7-0.8%.

In the invention, the implement of said three-dimensional crosslinking is preferred as: covering a film absorbed in metal cation solution to the surface of said solution after mixing and crosslinks to obtain said composite hydrogel. There are no special limitations to the material of the film or the covering process, routines that well known by the skilled in the art may be used.

In the invention, said crosslinking is optionally performed in sterile environment.

In the invention, said metal cations can react with alginate to form alginate hydrogel, the formed alginate hydrogel and acellular matrix hydrogel intertwines with each other to form a three-dimensional crosslinking structure with high density and stability, which further enhanced the adhesiveness of the composite hydrogel synergistically.

The composite hydrogel provided by the invention has controllable degradability and good mechanical strength, it is also easy to operate, and can seal the wound area in seconds.

The present invention also provides applications in donor cornea preparation, corneal adhesive, hydrogel dressing or drug carrier using said composite hydrogel or composite hydrogel prepared by said technical solutions. There are no special limitations on said applications, routines that well known by the skilled in the art may be used.

The composite hydrogel, when used as hydrogel dressing for ocular trauma, can promote fast healing of ocular surface epithelium; when used as donor cornea, can repair the corneal wounds effectively and maintain the integrity of the eyeball; when used as ophthalmic drug carrier, can achieve local drug administration with good sustainable release.

As to further describe the present invention, the figures and examples below will introduce the composite hydrogel and its preparation method and applications thereof provided by the invention in details, but they will not be understood as the limiting of the protection scope of the present invention.

### Example 1

Dissolve 0.3 U glutamine aminotransferase in 5 uL of 50 mmol/L trimethylaminomethane buffer solution (pH=7.4), and then mix with 150 ul of acellular matrix solution with mass concentration of 3% and 45 uL of dopamine modified sodium alginate solution with mass concentration of 4% to obtain a mixed solution. Cover the surface of the mixed solution with a film adsorbed with calcium chloride solution with concentration of 0.5 mol/L to obtain said composite hydrogel.

### Example 2

Dissolve 0.3 U glutamine aminotransferase in 5 uL of 50 mmol/L trimethylaminomethane buffer solution (pH=7.4), and then mix with 150 uL of acellular matrix solution with mass concentration of 3% and 45 uL of sodium alginate solution with mass concentration of 5% to obtain a mixed solution. Cover the surface of the mixed solution with a film adsorbed with calcium chloride solution with concentration of 0.5 mol/L to obtain said composite hydrogel.

### Application Example 1

After healthy New Zealand white rabbits being anesthetized, corneal stroma from the center of the rabbit's cornea is peeled off with a diameter of 6.25 mm and thickness of 200 um using trephine, and the corneal stroma defect model is prepared;

Inject precursor solution prepared in Example 1 onto a corneal contact lens with a diameter of 8 mm, immerse it in a 0.5 mol/L calcium chloride solution for 5 seconds, put the corneal contact lens on the ocular surface of rabbits for 2 seconds, remove the corneal contact lens, and then the composite hydrogel achieves to fill the corneal defect area;

Wash the corneal repair area with normal saline, put on corneal bandage lens to the defect eye, and send the New Zealand rabbits back to the feeding room for feeding. Within seven days after the operation, apply Tobramycin, Dexamethasone eye ointment every morning and evening in the defect area.

### Functional Tests

### Test Example 1

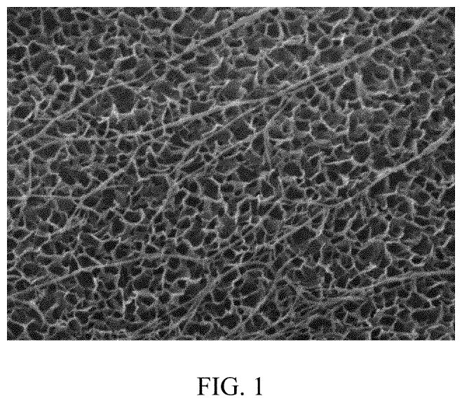

The composite hydrogel obtained in Example 1 is dried with supercritical carbon dioxide, and then observed and tested by a scanning electron microscope. The test results are shown in FIG. 1. It can be seen from FIG. 1 that the composite hydrogel obtained in this example presents a three-dimensional network structure.

### Test Example 2

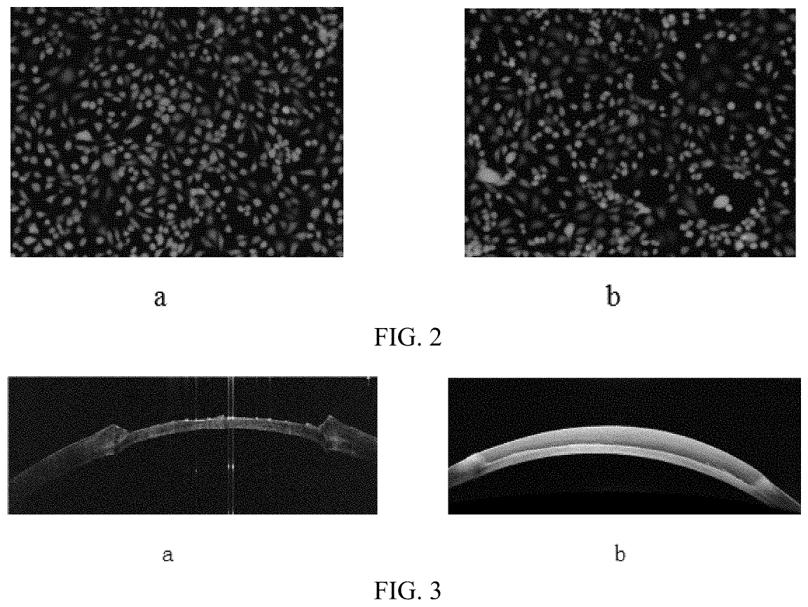

The composite hydrogel obtained in Example 1 is inoculated with human corneal epithelial cells. After three days of cell culture, live and dead cell fluorescence staining test is carried out. The test results are shown in FIG. 2, wherein a is the live and dead cell staining fluorescence test results of human corneal epithelial cells on the surface of the composite hydrogel, b is the live and dead cell staining fluorescence test results of human corneal epithelial cells on the culture dish, and red represents the dead cells, green represents the living cells. It can be seen from a that the survival rate of cells is over 98%, and from b that the survival rate of cells on the culture dish is 98.75%, indicating that the composite hydrogel obtained in this example has excellent biocompatibility.

### Test Example 3

On the day after surgery, optical coherence tomography (OCT) is performed on the corneal repair area in Application Example 1. The test conditions are: layer scan mode. The test results are shown in FIG. 3, wherein, a is the OCT scan of the unrepaired corneal defect area, and b is the OCT scan of the corneal defect area repaired by the composite hydrogel provided by the present invention. From b, it can be seen that the composite hydrogel provided by the present invention can repair the corneal stroma defect and restore normal refraction.

### Test Example 4

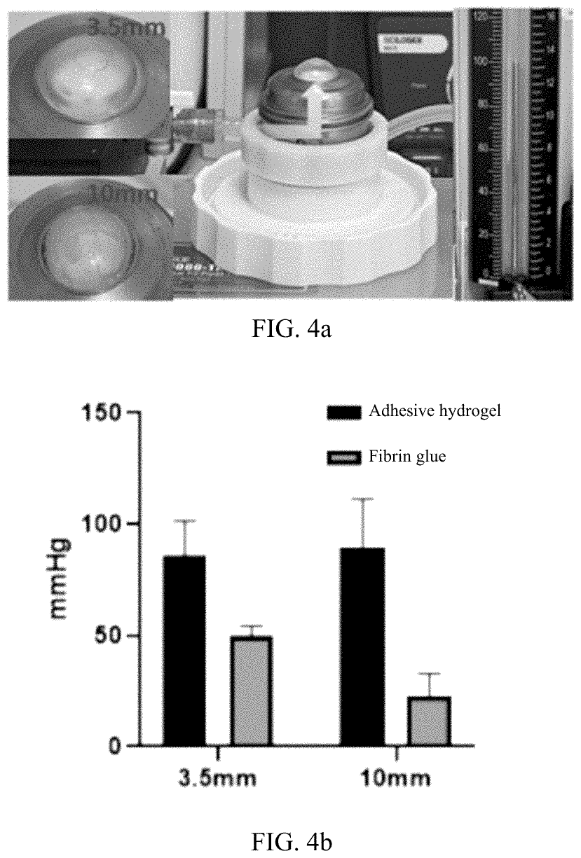

The maximum intraocular pressure sufferable of both the composite hydrogel obtained in Example 1 and the commercially available porcine fibrin glue (purchased from Guangzhou Beixiu Biotechnology Co., Ltd.) is tested. The method is to test with an artificial anterior chamber. The test device is shown in FIG. 4a. a shows an ex vivo 3.5 mm corneal perforation model and a 10 mm corneal cross incision model for testing both hydrogels. The test results are shown in FIG. 4b. It can be seen from b that the maximum intraocular pressure that the composite hydrogel (adhesive hydrogel) provided by the present invention can withstand is higher than that of the porcine fibrin adhesive, indicating that the composite hydrogel provided by the present invention has excellent adhesion and mechanical strength.

### Test Example 5

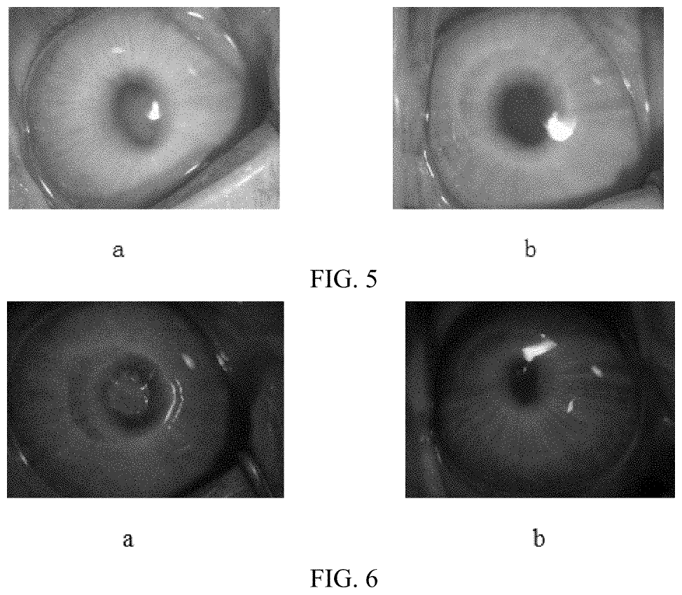

Three months after the operation, the corneal repair area in Application Example 1 was observed and tested by slit lamp. The test results are shown in FIG. 5, wherein, a is the general observation of the unrepaired cornea, and b is the general observation of the cornea repaired by the composite hydrogel. It can be seen from a and b that the corneal area repaired by the composite hydrogel provided by the present invention has no scar, while there is apparent nebula in the unrepaired corneal area.

### Test Example 6

Two weeks after the operation, the corneal repair area in Application Example 1 is stained with fluorescein sodium for observation. The method is as follows: the corneal repair area is stained, washed with normal saline, and photographed under cobalt blue light for observation. The test results are shown in FIG. 6, wherein, a is the sodium fluorescein staining result of the unrepaired cornea, and b is the sodium fluorescein staining result of the cornea repaired by the composite hydrogel. It can be seen from a and b that the corneal epithelium repaired by the composite hydrogel provided by the present invention is completely repaired, while there is an obvious epithelial defect in the center of the untreated cornea.

### Test Example 7

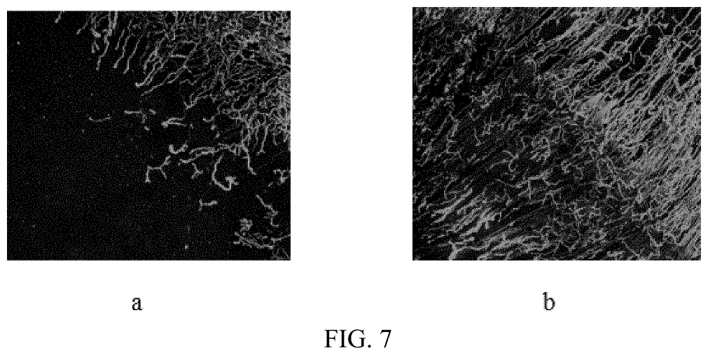

Six months after the operation, the corneal nerve staining test is performed on the corneal repair area in Application Example 1. The method is: immunofluorescence staining is performed on the repair area, and the corneal nerve is labeled with beta III tubulin antibody. The test results are shown in FIG. 7, wherein, a is the staining result of the unrepaired corneal nerve, and b is the staining result of the corneal nerve repaired by the composite hydrogel. It can be seen from a and b that the length and density of corneal nerve fibers repaired by the composite hydrogel provided by the present invention are significantly higher than those of untreated corneas.

Although the above embodiments make a detailed description of the present invention, but it is only a part of examples of the present invention, not all embodiments. Any other embodiments made by the skilled in the art based on the technical content disclosed in present embodiments without creative activities, fail within the protection scope of the present invention.

What is claimed is:

1. A composite hydrogel, comprises alginate hydrogel and acellular matrix hydrogel;
the ingredients of said acellular matrix hydrogel comprise acellular matrix and transglutaminases (TGases);
said alginate hydrogel comprises alginate complex;
said alginate hydrogel and acellular matrix hydrogel constitute a three-dimensional crosslinking structure.

2. The composite hydrogel according to claim 1, wherein the mass ratio of said alginate hydrogel and acellular matrix hydrogel is 1:1-4; the mass of said alginate hydrogel is calculated with the mass of alginates; the mass of said acellular matrix hydrogel is calculated with the mass of acellular matrix.

3. The composite hydrogel according to claim 1, wherein said alginate complex comprises alginate and metal cations, said alginate comprises one or more of unmodified alginate, peptide modified alginate and dopamine modified alginate.

4. The composite hydrogel according to claim 3, wherein said metal cations comprises one or more of calcium ions, magnesium ions and zinc ions.

5. A method for preparing a composite hydrogel, comprising:
after the mixing of acellular matrix solution, alginate solution, TGase, and buffer solution, using metal cation solution to promote three-dimensionally crosslinking and then said composite hydrogel is obtained.

6. The method according to claim 5, wherein the mass concentration of said acellular matrix solution is 1-3%;
the mass concentration of said alginate solution is 2-10%;
the volume ratio of said acellular matrix solution and alginate solution is 1-5:1.

7. The method according to claim 5, wherein the amount ratio of said TGase and acellular matrix in the acellular matrix solution is 20-100 U:1 g.

8. The method according to claim 7, wherein said buffer solution comprises phosphate buffer solution or trimethylaminomethane buffer solution;
the concentration of said buffer solution is 10-100 mmol/L;
the pH of said buffer solution is 6-8;
the volume of said buffer solution accounts for 1-5% of the volume of the acellular matrix solution.

9. The method according to claim 5, wherein the molar concentration of said metal cations in said metal cation solution is 0.2-5 mol/L;
the volume of said metal ion solution accounts for 0.5-1% of the volume of alginate solution.

10. The method according to claim 5, wherein said crosslinking is performed in sterile environment.

11. Applications of said composite hydrogel in claim 1 in preparing donor cornea, corneal adhesive, hydrogel dressing or drug carrier.

12. The applications according to claim 11, wherein said application in preparing donor cornea comprises:
mixing acellular matrix solution, alginate solution, glutamine aminotransferase, and buffer solution to obtain a mixture solution; injecting said mixture solution onto corneal contact lens and immersing it in metal cation solution; then covering corneal defect area with said corneal contact lens to achieves the filling of the corneal defect area with the composite hydrogel.

13. The applications according to claim 12, wherein the mass concentration of said acellular matrix solution is 1-3%;
the mass concentration of said alginate solution is 2-10%;
the volume ratio of said acellular matrix solution and said alginate solution is 1-5:1.

14. The applications according to claim 12, wherein the amount ratio of said TGase and acellular matrix in said acellular matrix solution is 20-100 U:1 g.

15. The applications according to claim 12, wherein said buffer solution comprises phosphate buffer solution or TRIS buffer solution;
the concentration of said buffer solution is 10-100 mmol/L;
the pH value of said buffer solution is 6-8;
the volume of said buffer solution accounts for 1-5% of the volume of said acellular matrix solution.

16. The applications according to claim 12, wherein the molar concentration of metal cations in said metal cation solution is 0.2-5 mol/L;
the volume ratio of said metal cation solution accounts for in the alginate solution is 0.5-1%.

17. The applications according to claim 12, wherein the diameter of said corneal contact lens is 8 mm.

18. The applications according to claim 12, wherein said immersing time is 5 seconds.

19. The applications according to claim 12, wherein said covering time is 2 seconds.
